(19) United States
(12) Patent Application Publication
(10) Pub. No.: US 2026/0127738 A1
(43) Pub. Date: May 7, 2026

(54) EMOTION IDENTIFICATION OF INDIVIDUALS

(71) Applicant: Texas State University, San Marcos, TX (US)

(72) Inventors: Maria Resendiz, Austin, TX (US); Damian Valles, San Marcos, TX (US); Md Inzamam Ul Haque, Bothell, WA (US); Rezwan Matin, San Marcos, TX (US); Tamima Rashid, Austin, TX (US)

(21) Appl. No.: 19/378,888

(22) Filed: Nov. 4, 2025

Related U.S. Application Data
(60) Provisional application No. 63/716,320, filed on Nov. 5, 2024.

(51) Int. Cl. G06T 7/00; G16H 50/20

(52) U.S. Cl. CPC G06T 7/0012; G16H 50/20; G06T 2207/20081; G06T 2207/20084; G06T 2207/30201

(57) ABSTRACT

A computer-implemented method, system, and computer program product for emotion recognition. A deep convolutional neural network is trained to identify an emotion from images of facial expressions. Furthermore, an application in a computing device (e.g., mobile computing device, such as a smartphone) is utilized to capture an image of an individual (e.g., individual that is neurotypical, individual on the autism spectrum). The captured image of the individual is then analyzed using the trained deep convolutional neural network. The emotional state of the individual is then classified based on the analysis of the captured image of the individual using the trained deep convolutional neural network. The classified emotional state is then conveyed to a user (e.g., neurotypical individual, autistic individual) via an emoticon (emotional icon), such that the emoticon reflects the classified emotional state.

### GOVERNMENT INTERESTS

This invention was made with government support under Grant Numbers 2150135 and 2231794 awarded by the National Science Foundation. The government has certain rights in the invention.

### TECHNICAL FIELD

The present disclosure relates generally to emotion recognition, and more particularly to identifying emotions of individuals, including individuals on the autism spectrum.

### BACKGROUND

Emotion recognition is the process of identifying human emotion. People vary widely in their accuracy at recognizing the emotions of others. Use of technology to help people with emotion recognition is a relatively nascent research area. Generally, the most effective systems employ a multimodal approach, such as by analyzing various human expressions in context. For example, existing techniques focus on automating the recognition of facial expressions from video, spoken expressions from audio, written expressions from text, and physiology as measured by wearables.

The accuracy of emotion recognition is usually improved when it combines the analysis of human expressions from multimodal forms, such as texts, physiology, audio, or video. Different emotion types are detected through the integration of information from facial expressions, body movement and gestures, and speech. The technology is said to contribute in the emergence of the so-called emotional or emotive Internet.

The existing approaches in emotion recognition to classify certain emotion types can be generally classified into three main categories: knowledge-based techniques, statistical methods, and hybrid approaches.

Unfortunately, the developmental process for these existing emotion recognition and teaching technologies fails to include the autistic perspective. Autism spectrum disorder (ASD) is a neurodevelopmental condition marked by challenges in social communication and a tendency towards repetitive, restrictive patterns of behavior and interests. Furthermore, ASD involves autistic individuals having a range of support needs.

There is inconclusive information regarding how autistic individuals interpret and learn emotions. As a result, existing technological models are built without this crucial data making them largely neurotypical-centric.

Hence, there is not currently a means for bidirectional teaching to provide information to neurotypical individuals about how autistic individuals learn emotions and vice-versa.

### SUMMARY

In one embodiment of the present disclosure, a computer-implemented method for emotion recognition comprises training a deep convolutional neural network to identify an emotion from images of facial expressions. The method further comprises capturing an image of an individual from a computing device. The method additionally comprises analyzing the captured image of the individual using the trained deep convolutional neural network. Furthermore, the method comprises classifying an emotional state of the individual based on the analysis of the captured image of the individual using the trained deep convolutional neural network. Additionally, the method comprises conveying the classified emotional state to a user of the computing device via an emoticon.

Other forms of the embodiment of the computer-implemented method described above are in a system and in a computer program product.

The foregoing has outlined rather generally the features and technical advantages of one or more embodiments of the present disclosure in order that the detailed description of the present disclosure that follows may be better understood. Additional features and advantages of the present disclosure will be described hereinafter which may form the subject of the claims of the present disclosure.

### BRIEF DESCRIPTION OF THE DRAWINGS

A better understanding of the present disclosure can be obtained when the following detailed description is considered in conjunction with the following drawings, in which:

FIG. 1 illustrates an embodiment of the present disclosure of a computing environment for practicing the principles of the present disclosure;

FIG. 2 is a diagram of the software components used by the computer to identify emotions of individuals, including individuals on the autism spectrum, in accordance with an embodiment of the present disclosure; and

FIG. 3 is a flowchart of a method for assisting neurotypical people in identifying emotions from non-neurotypical people (e.g., people with ASD) and vice-versa in accordance with an embodiment of the present disclosure.

### DETAILED DESCRIPTION

As stated above, emotion recognition is the process of identifying human emotion. People vary widely in their accuracy at recognizing the emotions of others. Use of technology to help people with emotion recognition is a relatively nascent research area. Generally, the most effective systems employ a multimodal approach, such as by analyzing various human expressions in context. For example, existing techniques focus on automating the recognition of facial expressions from video, spoken expressions from audio, written expressions from text, and physiology as measured by wearables.

The accuracy of emotion recognition is usually improved when it combines the analysis of human expressions from multimodal forms, such as texts, physiology, audio, or video. Different emotion types are detected through the integration of information from facial expressions, body movement and gestures, and speech. The technology is said to contribute in the emergence of the so-called emotional or emotive Internet.

The existing approaches in emotion recognition to classify certain emotion types can be generally classified into three main categories: knowledge-based techniques, statistical methods, and hybrid approaches.

Unfortunately, the developmental process for these existing emotion recognition and teaching technologies fails to include the autistic perspective. Autism spectrum disorder (ASD) is a neurodevelopmental condition marked by challenges in social communication and a tendency towards repetitive, restrictive patterns of behavior and interests. Furthermore, ASD involves autistic individuals having a range of support needs.

There is inconclusive information regarding how autistic individuals interpret and learn emotions. As a result, existing technological models are built without this crucial data making them largely neurotypical-centric.

Hence, there is not currently a means for bidirectional teaching to provide information to neurotypical individuals about how autistic individuals learn emotions and vice-versa.

The embodiments of the present disclosure provide a means for identifying emotions that include both the neurotypical and autistic perspective. In one embodiment, a deep convolutional neural network is trained to identify an emotion from images of facial expressions. For example, the deep convolutional neural network (deep CNN) is trained on a sample data set that includes images of individuals expressing seven emotions (e.g., anger, contempt, disgust, fear, happiness, sadness, surprise) photographed from five different angles. Examples of such a convolutional neural network include Naïve-CNN, VGG16, EfficientNetV2, and MobileNetV2. In one embodiment, an application in a computing device (e.g., mobile computing device, such as a smartphone) is utilized to capture an image of an individual (e.g., individual that is neurotypical, individual on the autism spectrum). The captured image of the individual is then analyzed using the trained deep convolutional neural network. The emotional state of the individual is then classified, such as one of the seven emotions discussed above, based on the analysis of the captured image of the individual using the trained deep convolutional neural network. The classified emotional state is then conveyed to a user (e.g., individual on the autism spectrum, individual that is neurotypical) via an emoticon (emotional icon), such that the emoticon reflects the classified emotional state. In this manner, the emotion of an individual, such as an individual on the autism spectrum, is recognized for a user, such as an individual that is neurotypical and vice-versa. A further discussion regarding these and other features is provided below.

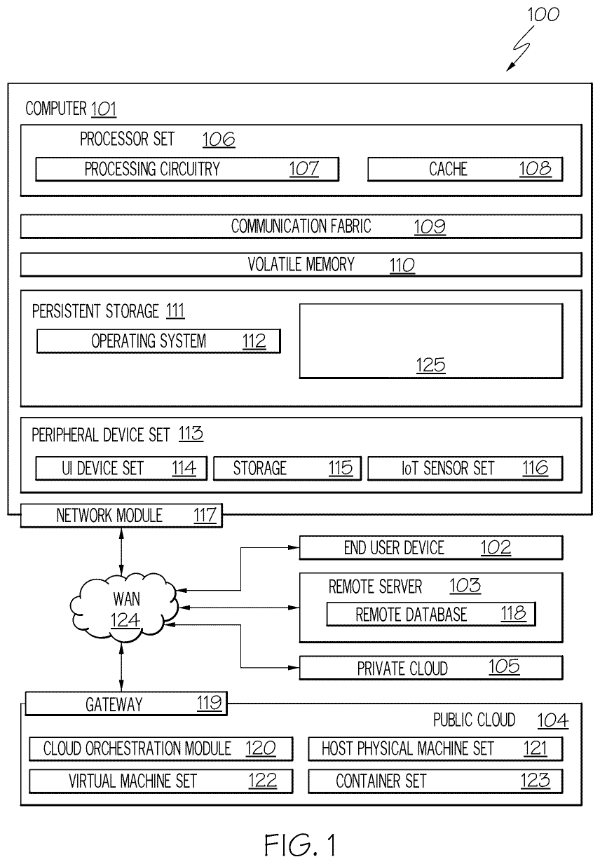

Referring now to the Figures in detail, FIG. 1 illustrates an embodiment of the present disclosure of a computing environment 100 for practicing the principles of the present disclosure.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code (stored in block 125) involved in performing the inventive methods, such as assisting neurotypical people in identifying emotions from non-neurotypical people (e.g., people with ASD) and vice-versa. In addition to block 125, computing environment 100 includes, for example, computer 101 (also referred to herein as computing device 101), network 124, such as a wide area network (WAN), end user device (EUD) 102, remote server 103, public cloud 104, and private cloud 105. In this embodiment, computer 101 includes processor set 106 (including processing circuitry 107 and cache 108), communication fabric 109, volatile memory 110, persistent storage 111 (including operating system 112 and block 125, as identified above), peripheral device set 113 (including user interface (UI) device set 114, storage 115, and Internet of Things (IoT) sensor set 116), and network module 117. Remote server 103 includes remote database 118. Public cloud 104 includes gateway 119, cloud orchestration module 120, host physical machine set 121, virtual machine set 122, and container set 123.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 118. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor set 106 includes one, or more, computer processors of any type now known or to be developed in the future. Processing circuitry 107 may be distributed over multiple packages, for example, multiple, coordinated integrated circuit chips. Processing circuitry 107 may implement multiple processor threads and/or multiple processor cores. Cache 108 is memory that is located in the processor chip package(s) and is typically used for data or code that should be available for rapid access by the threads or cores running on processor set 106. Cache memories are typically organized into multiple levels depending upon relative proximity to the processing circuitry. Alternatively, some, or all, of the cache for the processor set may be located “off chip.” In some computing environments, processor set 106 may be designed for working with qubits and performing quantum computing.

Computer readable program instructions are typically loaded onto computer 101 to cause a series of operational steps to be performed by processor set 106 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 108 and the other storage media discussed below. The program instructions, and associated data, are accessed by processor set 106 to control and direct performance of the inventive methods. In computing environment 100, at least some of the instructions for performing the inventive methods may be stored in block 125 in persistent storage 111.

Communication fabric 109 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 110 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 110 is located in a single package and is internal to computer 101, but, alternatively or additionally, the volatile memory may be distributed over multiple packages and/or located externally with respect to computer 101.

Persistent Storage 111 is any form of non-volatile storage for computers that is now known or to be developed in the future. The non-volatility of this storage means that the stored data is maintained regardless of whether power is being supplied to computer 101 and/or directly to persistent storage 111. Persistent storage 111 may be a read only memory (ROM), but typically at least a portion of the persistent storage allows writing of data, deletion of data and re-writing of data. Some familiar forms of persistent storage include magnetic disks and solid state storage devices. Operating system 112 may take several forms, such as various known proprietary operating systems or open source Portable Operating System Interface type operating systems that employ a kernel. The code included in block 125 typically includes at least some of the computer code involved in performing the inventive methods.

Peripheral device set 113 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 114 may include components such as a display screen, speaker, microphone, wearable devices (such as goggles and smart watches), keyboard, mouse, printer, touchpad, game controllers, and haptic devices. Storage 115 is external storage, such as an external hard drive, or insertable storage, such as an SD card. Storage 115 may be persistent and/or volatile. In some embodiments, storage 115 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 116 is made up of sensors that can be used in Internet of Things applications. For example, one sensor may be a thermometer and another sensor may be a motion detector.

Network module 117 is the collection of computer software, hardware, and firmware that allows computer 101 to communicate with other computers through WAN 124. Network module 117 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 117 are performed on the same physical hardware device. In other embodiments (for example, embodiments that utilize software-defined networking (SDN)), the control functions and the forwarding functions of network module 117 are performed on physically separate devices, such that the control functions manage several different network hardware devices. Computer readable program instructions for performing the inventive methods can typically be downloaded to computer 101 from an external computer or external storage device through a network adapter card or network interface included in network module 117.

WAN 124 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 102 is any computer system that is used and controlled by an end user (for example, a customer of an enterprise that operates computer 101), and may take any of the forms discussed above in connection with computer 101. EUD 102 typically receives helpful and useful data from the operations of computer 101. For example, in a hypothetical case where computer 101 is designed to provide a recommendation to an end user, this recommendation would typically be communicated from network module 117 of computer 101 through WAN 124 to EUD 102. In this way, EUD 102 can display, or otherwise present, the recommendation to an end user. In some embodiments, EUD 102 may be a client device, such as thin client, heavy client, mainframe computer, desktop computer and so on.

Remote server 103 is any computer system that serves at least some data and/or functionality to computer 101. Remote server 103 may be controlled and used by the same entity that operates computer 101. Remote server 103 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 118 of remote server 103.

Public cloud 104 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 104 is performed by the computer hardware and/or software of cloud orchestration module 120. The computing resources provided by public cloud 104 are typically implemented by virtual computing environments that run on various computers making up the computers of host physical machine set 121, which is the universe of physical computers in and/or available to public cloud 104. The virtual computing environments (VCEs) typically take the form of virtual machines from virtual machine set 122 and/or containers from container set 123. It is understood that these VCEs may be stored as images and may be transferred among and between the various physical machine hosts, either as images or after instantiation of the VCE. Cloud orchestration module 120 manages the transfer and storage of images, deploys new instantiations of VCEs and manages active instantiations of VCE deployments. Gateway 119 is the collection of computer software, hardware, and firmware that allows public cloud 104 to communicate through WAN 124.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 105 is similar to public cloud 104, except that the computing resources are only available for use by a single enterprise. While private cloud 105 is depicted as being in communication with WAN 124 in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 104 and private cloud 105 are both part of a larger hybrid cloud.

Block 125 further includes the software components which are used to assist neurotypical people in identifying emotions from non-neurotypical people (e.g., people with ASD) and vice-versa. In one embodiment, such components may be implemented in hardware. The functions discussed above performed by such components are not generic computer functions. As a result, computer 101 is a particular machine that is the result of implementing specific, non-generic computer functions.

In one embodiment, the functionality of such software components of computer 101, including the functionality for assisting neurotypical people in identifying emotions from non-neurotypical people (e.g., people with ASD) and vice-versa, may be embodied in an application specific integrated circuit.

A discussion regarding the principles of the present disclosure identifying emotions from non-neurotypical people (e.g., people with ASD) and vice-versa is provided below.

In one embodiment, the principles of the present disclosure are embodied in an application (“the emotion identification experience app”), which is used as a tool to identify the emotion of a person. In one embodiment, the emotion identification experience app is used as a teaching tool for social emotion learning of how people learn about and identify emotions. Perspectives of both neurotypical individuals and autistic individuals are considered.

In one embodiment, the emotion expression recognition app aids children with learning about how people on the autism spectrum learn, utilizing deep learning techniques for processing and classifying emotion expressions in real time.

In one embodiment, such deep learning models utilized include Naïve-CNN, VGG16, EfficientNetV2, and MobileNetV2, which were trained using datasets from FER2013 and Zoom video recordings to focus on facial expression recognition. In one embodiment, preprocessing techniques, such as histogram equalization, brightness and contrast adjustments, and augmentation, are applied to improve model accuracy.

In one embodiment, the data is collected via Zoom® interviews involving 208 participants, focusing on seven universal emotions (e.g., happy, sad, angry, fearful, neutral, surprised, and disgust). In one embodiment, data curation involved processing video clips to isolate facial expressions, followed by a labeling process to ensure accurate emotion classification.

In one embodiment, an iOS app is used to deploy these models, enabling real-time facial emotion recognition using the camera. In one embodiment, the app's interface is designed for simplicity, allowing users to capture facial expressions and receive instant feedback in emoticons representing the detected emotion.

In one embodiment, the emotion identification experience app uses facial, voice, and gestural information to identify an emotion.

In one embodiment, the emotion identification experience app is used as a tool that can be used to teach neurotypical people about how autistic individuals learn emotions and to autistic people about how neurotypical people learn about emotions.

In one embodiment, the principles of the present disclosure are directed to a mobile app tool designed to automatically discern a person's emotional state by interpreting the nuances of facial expressions and translating these into identifiable emotional states. Such an application will aid individuals on the autism spectrum, counselors, and educators in learning to recognize the emotions of others and help improve social interactions.

In one embodiment, a deep convolutional neural network (DCNN), such as Naïve-CNN, VGG16, EfficientNetV2, and MobileNetV2, is trained to identify an emotion from images of facial expressions. In one embodiment, the DCNN is trained on a dataset of images (e.g., 4,900 images) of individuals (e.g., 70 individuals) expressing seven emotions photographed from different angles (e.g., five different angles), enabling it to detect emotions with an accuracy exceeding 80%.

In one embodiment, a mobile application runs the designated and accompanying image-processing algorithms. Utilizing the mobile device's camera lens, the targeted individual's image is captured and analyzed through the DCNN, which classifies the emotional state detected. The identified emotion is then conveyed to the user through an emoticon, offering a visual representation of the emotional state of the person in focus.

Benefits of utilizing such technology include being able to analyze complex data from facial expressions with high accuracy in real-time.

Furthermore, in one embodiment, the principles of the present disclosure operate via a mobile application, making it non-invasive and easily accessible to a wide user base. This ease of use extends to everyday environments, making continuous support in real-world settings practical.

Additionally, in one embodiment, the principles of the present disclosure address the challenges autistic individuals face by having the application aid in recognizing and understanding emotional cues, a critical area of need.

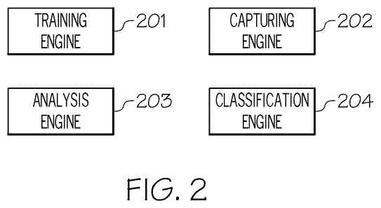

A further discussion regarding the functionality of the components used by computer 101 to identify emotions of individuals, including individuals on the autism spectrum, is provided below in connection with FIG. 2.

FIG. 2 is a diagram of the software components used by computer 101 to identify emotions of individuals, including individuals on the autism spectrum, in accordance with an embodiment of the present disclosure.

Referring to FIG. 2, in conjunction with FIG. 1, computer 101 includes training engine 201 configured to train a deep convolutional neural network to identify an emotion from images of facial expressions.

In one embodiment, training engine 201 trains a deep convolutional neural network to identify an emotion from images of facial expressions by implementing a multi-step process, involving data preparation, model selection, preprocessing, training, and evaluation.

In connection with data preparation, in one embodiment, training engine 201 acquires and prepares a large, labeled dataset of facial expression images. In one embodiment, the data source includes a public benchmark dataset used for facial emotion recognition (e.g., facial express recognition (FER) 2013). In one embodiment, the data source includes video data (e.g., Zoom® video data) that was collected and processed to isolate facial expressions and ensure accurate emotion classification, covering the seven universal emotions (e.g., happy, sad, angry, fearful, neutral, surprised, and disgust).

Furthermore, in connection with data preparation, in one embodiment, training engine 201 performs data curation and labeling. In one embodiment, data curation and labeling involves processing video clips to extract still images or short sequences of facial expressions followed by labeling each image or sequence with the corresponding emotional state (e.g., “happy,” “angry”). In one embodiment, training engine 201 performs such data curation and labeling using a pre-trained, general-purpose facial emotion recognition (FER) model to process large volumes of unlabeled data (e.g., Zoom® video recordings). In one embodiment, training engine 201 runs the video clips or images through the pre-trained FER model, which assign an emotion label (e.g., “happy” with a 90% confidence, “neutral” with an 8% confidence) to each frame or image. This creates a pre-labeled dataset.

In one embodiment, training engine 201 implements high-confidence filtering which accepts and labels data points where the pre-trained model's confidence is very high (e.g., above 95%). On the other hand, data points with low confidence or ambiguous predictions are flagged for manual review.

Additionally, in connection with data preparation, in one embodiment, training engine 201 performs data augmentation. For example, training engine 201 implements preprocessing techniques, such as data augmentation, which artificially increases the dataset size by creating modified copies of images (e.g., flipping, rotating, zooming) to help the model generalize better and prevent overfitting.

In one embodiment, training engine 201 selects one of the deep convolutional neural network models for image analysis, such as Naïve-CNN, VGG16, EfficientNetV2, or MobileNetV2. In one embodiment, the selected deep convolutional neural network model consists of multiple levels, such as convolutional layers (apply filters to the input image to automatically learn hierarchical feature representations (edges, textures, shapes)), pooling layers (these reduce the spatial dimensions of the feature maps, making the model more robust to minor variations in face position), fully connected layers (these take the high-level features learned by the convolutional layers and use them to make the final classification), and output layer (this layer uses a softmax function to produce a probability distribution over the seven emotion classes, indicating the model's confidence for each emotion).

In one embodiment, training engine 201 performs preprocessing by applying preprocessing techniques (e.g., histogram equalization, brightness/contrast adjustments) to standardize the data and improve model performance prior to feeding images into the deep convolutional neural network.

In one embodiment, training engine 201 uses a categorical cross-entropy loss function to quantify the difference between the model's predicted emotion probabilities and the true emotion label.

In one embodiment, training engine 201 uses an optimizer (e.g., Adam, SGD) to minimize the loss function by iteratively updating the model's weights during backpropagation.

In one embodiment, training engine 201 trains the deep convolutional neural network to identify an emotion from images of facial expression over multiple passes (epochs) through the entire dataset. In each pass, a batch of images is fed forward, the loss is calculated, and the weights are updated backward.

In one embodiment, training engine 201 evaluates the performance of the model (e.g., Naïve-CNN, VGG16) using metrics, such as accuracy, precision, recall, and F1-score (harmonic mean of a model's precision and recall).

In one embodiment, training engine 201 fine-tunes the deep convolutional neural network model for optimal performance. For example, training engine 201 adjusts the hyperparameters (e.g., learning rate, batch size, number of layers) or applies advanced techniques, such as transfer learning, to optimize the model. In one embodiment, training engine 201 retrains the model to improve emoticon performance. In one embodiment, the resulting trained deep convolutional neural network model is incorporated into an application, such as an iOS application.

In one embodiment, training engine 201 trains the deep convolutional neural network model to identify an emotion from images of facial expressions based on a sample data set, which may include labeled emotions for various images of facial expressions. In one embodiment, such labeled emotions (sample data set) are obtained from an expert or a pre-trained FER model. In one embodiment, the sample data set includes images of individuals expressing seven emotions photographed from five different angles.

Furthermore, in one embodiment, the sample data set discussed above is referred to herein as the “training data,” which is used by a machine learning algorithm to make predictions or decisions, such as identifying an emotion from images of facial expressions. The algorithm iteratively makes predictions on the training data until the predictions achieve the desired accuracy as determined by an expert. Examples of such learning algorithms include neural networks.

Computer 101 further includes capturing engine 202 configured to capture an image of an individual from computing device 101, which, in one embodiment, corresponds to a mobile computing device.

In one embodiment, capturing engine 202 manages the camera feed of the computing device's (e.g., mobile computing device) camera hardware and saves the resulting image data.

In one embodiment, capturing engine 202 uses a framework (e.g., AVFoundation framework), which provides the necessary application programming interfaces (APIs) for interacting with the computing device's cameras.

In one embodiment, capturing engine 202 determines which camera on computer/computing device 101 (e.g., mobile computing device) to use. For facial emotion recognition (FER), in one embodiment, capturing engine 202 selects the front-facing camera to capture the individual using computing device 101.

In one embodiment, capturing engine 202 establishes a capture session using the AVFoundation framework, linking the selected camera input to a data output.

In one embodiment, capturing engine 202 displays a live camera feed on the computing device's screen (e.g., within the app interface). This allows the user to position their face correctly within the frame for optimal analysis.

In one embodiment, capturing engine 202 then captures a still image or extracts a video frame from the live stream.

In one embodiment, capturing engine 202 delivers the captured image as digital data (e.g., a UIImage object or raw pixel buffer) to analysis engine 203.

In one embodiment, capturing engine 202 uses a specific camera-related function (e.g., capturePhoto(with:delegate:) in iOS) to grab the image data. The resulting image is then cropped or processed to isolate the face before being passed to the trained Deep Convolutional Neural Network (DCNN) for analysis.

Computer 101 additionally includes analysis engine 203 configured to analyze the captured image of the individual, which, in one embodiment, corresponds to an individual on the autism spectrum or an individual that is neurotypical, using the trained deep convolutional neural network.

In one embodiment, analysis engine 203 performs pre-processing and formatting. In one embodiment, the raw image captured by the camera is converted into a specific format and size required by the trained deep convolutional neural network model.

In one embodiment, analysis engine 203 receives the captured image data (e.g., a UIImage) from capturing engine 202.

In one embodiment, before emotion analysis, analysis engine 203 identifies the location of the face within the image. In one embodiment, such an individuation involves analysis engine 203 using a separate, fast face detection algorithm to output bounding box coordinates for the face.

In one embodiment, analysis engine 203 crops the image to focus only on the face. In one embodiment, analysis engine 203 resizes the cropped image to be the exact input dimensions that the trained deep convolutional neural network model expects (e.g., input dimension in pixels).

In one embodiment, analysis engine 203 normalizes (scales) the pixel values of the resized image to match the range of values used during the model's training phase (e.g., scaling pixel values from 0 to 255 to a floating-point range, such as 0 to 1 or −1 to 1).

In one embodiment, analysis engine 203 loads the pre-trained and optimized deep convolutional neural network into memory of computing device 101. On iOS devices, analysis engine 203 performs such a task using a machine learning framework, such as Core ML (for model integration and inference) or metal performance shaders (for hardware-accelerated computations).

In one embodiment, analysis engine 203 feeds the pre-processed, formatted image data into the deep convolutional neural network. In one embodiment, analysis engine 203 performs all the forward passes through the network's layers (convolutional, pooling, fully connected) as defined during the training process.

In one embodiment, the deep convolutional neural network produces an output vector from its final (softmax) layer. In one embodiment, such a vector contains a probability score for each of the seven universal emotion classes (e.g., happy, sad, angry, fearful, neutral, surprised, and disgust).

In one embodiment, analysis engine 203 interprets the model's output to prepare for classification. In one embodiment, analysis engine 203 reads the probability vector to determine the emotional state. For example, if the seven emotions are ordered as (anger, disgust, fear, happy, sad, surprise, neutral), and the vector is (0.02, 0.01, 0.03, 0.90, 0.01, 0.02, 0.01), then analysis engine 203 identifies happy as the most likely emotion with a 90% confidence.

In one embodiment, analysis engine 203 passes the prediction (both the chosen emotion and its confidence score) to classification engine 204, which is configured to classify the emotional state of the individual based on the analysis of the captured image of the individual using the trained deep convolutional neural network.

In one embodiment, classification engine 204 converts the deep convolutional neural network model's output into a discrete, human-interpretable emotion label.

In one embodiment, classification engine 204 identifies the output vector. In one embodiment, the deep convolutional neural network's final layer (the softmax layer) produces an output vector (a list of numbers) where each value corresponds to the probability of the image belonging to one of the target emotion classes (e.g., happy, sad, angry, etc.). The sum of all probabilities in this vector equals 1.0 (or 100%).

For example, a seven-element output vector might look like this:
Output Vector=[0.05(angry), 0.92(happy), 0.01(sad), 0.02(neutral), . . . ]

In one embodiment, classification engine 204 uses the argmax (argument of the maximum) function for classification. In one embodiment, the argmax function finds the index of the highest probability value in the output vector. Classification engine 204 then iterates through the vector and identifies the class associated with the maximum probability.

In the example above, the highest probability is 0.92, which corresponds to the happy class. Therefore, the classified emotional state is happy.

In one embodiment, classification engine 204 implements a confidence threshold to handle ambiguous or uncertain classifications. For example, if the maximum probability (Pₘₐₓ) meets or exceeds a defined threshold (e.g., 75%), then the corresponding emotion is confidently classified. In another example, if the maximum probability is below the threshold, the classification is flagged as ambiguous or uncertain (e.g., “cannot determine emotion”), rather than making a low-confidence guess.

In one embodiment, classification engine 204 outputs the final output of this stage, which is a single, discrete, text-based or numerical label (e.g., “happy”) that represents the emotional state.

In one embodiment, classification engine 204 conveys the classified emotional state to a user, which, in one embodiment, corresponds to an individual on the autism spectrum or an individual that is neurotypical, of computing device 101 via an emoticon.

In one embodiment, classification engine 204 conveys the classified emotional state to a user of computing device 101 via an emoticon by utilizing a mapping and display process, which may be handled by an application's user interface (UI) module.

In one embodiment, classification engine 204 maps the output (e.g., a discrete emotional state label, such as “happy”) to a corresponding visual element (the emoticon). In one embodiment, such a mapping is stored in a data structure, referred to herein as the “emoticon mapping table.” In one embodiment, such a data structure is stored in a storage device (e.g., storage device 113, 115) of computing device 101. In one embodiment, the data structure is populated by an expert.

In one embodiment, a dictionary or key-value array is used to pair the text-based classification with a specific emoticon or image asset. For example, the classified emotional state (input) is paired with an emoticon/asset (output). For instance, “happy” is paired with an emoticon of a happy graphic, “sad” is paired with an emoticon of a sad graphic, “angry” is paired with an emoticon of an angry graphic, “fearful” is paired with an emoticon of a fearful graphic, “neutral” is paired with an emoticon of a neutral graphic, “surprised” is paired with an emoticon of a surprised graphic, “disgust” is paired with an emoticon of a disgusted graphic, etc.

In one embodiment, the application's user interface component on computing device 101 is responsible for taking the selected emoticon and displaying it to the user of computing device 101. For example, the user interface component receives the classified state (e.g., happy) from classification engine 204. The user interface component then queries the emoticon mapping table to retrieve the corresponding visual asset (e.g., the code for the corresponding emoticon or the file path for the emoticon). In one embodiment, the application on computing device 101 then dynamically updates a designated area of the computing device's display (the user interface) by rendering the area with the retrieved emoticon.

In this manner, emotions of individuals, including individuals on the autism spectrum, are identified.

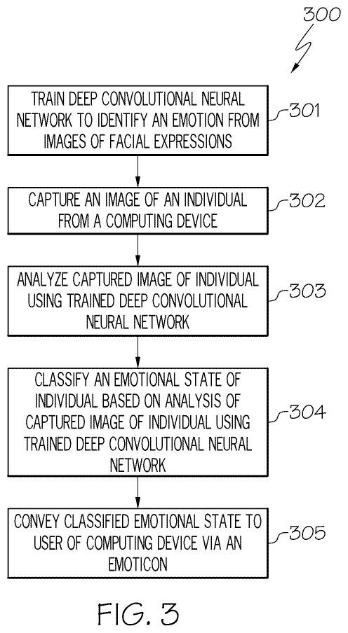

A discussion regarding the method for assisting neurotypical people in identifying emotions from non-neurotypical people (e.g., people with ASD) and vice-versa is provided below in connection with FIG. 3.

FIG. 3 is a flowchart of a method 300 for assisting neurotypical people in identifying emotions from non-neurotypical people (e.g., people with ASD) and vice-versa in accordance with an embodiment of the present disclosure.

Referring to FIG. 3, in conjunction with FIGS. 1-2, in step 301, training engine 201 trains a deep convolutional neural network to identify an emotion from images of facial expressions.

As stated above, in one embodiment, training engine 201 trains a deep convolutional neural network to identify an emotion from images of facial expressions by implementing a multi-step process, involving data preparation, model selection, preprocessing, training, and evaluation.

In connection with data preparation, in one embodiment, training engine 201 acquires and prepares a large, labeled dataset of facial expression images. In one embodiment, the data source includes a public benchmark dataset used for facial emotion recognition (e.g., facial express recognition (FER) 2013). In one embodiment, the data source includes video data (e.g., Zoom® video data) that was collected and processed to isolate facial expressions and ensure accurate emotion classification, covering the seven universal emotions (e.g., happy, sad, angry, fearful, neutral, surprised, and disgust).

Furthermore, in connection with data preparation, in one embodiment, training engine 201 performs data curation and labeling. In one embodiment, data curation and labeling involves processing video clips to extract still images or short sequences of facial expressions followed by labeling each image or sequence with the corresponding emotional state (e.g., “happy,” “angry”). In one embodiment, training engine 201 performs such data curation and labeling using a pre-trained, general-purpose facial emotion recognition (FER) model to process large volumes of unlabeled data (e.g., Zoom® video recordings). In one embodiment, training engine 201 runs the video clips or images through the pre-trained FER model, which assign an emotion label (e.g., “happy” with a 90% confidence, “neutral” with an 8% confidence) to each frame or image. This creates a pre-labeled dataset.

In one embodiment, training engine 201 implements high-confidence filtering which accepts and labels data points where the pre-trained model's confidence is very high (e.g., above 95%). On the other hand, data points with low confidence or ambiguous predictions are flagged for manual review.

Additionally, in connection with data preparation, in one embodiment, training engine 201 performs data augmentation. For example, training engine 201 implements preprocessing techniques, such as data augmentation, which artificially increases the dataset size by creating modified copies of images (e.g., flipping, rotating, zooming) to help the model generalize better and prevent overfitting.

In one embodiment, training engine 201 selects one of the deep convolutional neural network models for image analysis, such as Naïve-CNN, VGG16, EfficientNetV2, or MobileNetV2. In one embodiment, the selected deep convolutional neural network model consists of multiple levels, such as convolutional layers (apply filters to the input image to automatically learn hierarchical feature representations (edges, textures, shapes)), pooling layers (these reduce the spatial dimensions of the feature maps, making the model more robust to minor variations in face position), fully connected layers (these take the high-level features learned by the convolutional layers and use them to make the final classification), and output layer (this layer uses a softmax function to produce a probability distribution over the seven emotion classes, indicating the model's confidence for each emotion).

In one embodiment, training engine 201 performs preprocessing by applying preprocessing techniques (e.g., histogram equalization, brightness/contrast adjustments) to standardize the data and improve model performance prior to feeding images into the deep convolutional neural network.

In one embodiment, training engine 201 uses a categorical cross-entropy loss function to quantify the difference between the model's predicted emotion probabilities and the true emotion label.

In one embodiment, training engine 201 uses an optimizer (e.g., Adam, SGD) to minimize the loss function by iteratively updating the model's weights during backpropagation.

In one embodiment, training engine 201 trains the deep convolutional neural network to identify an emotion from images of facial expression over multiple passes (epochs) through the entire dataset. In each pass, a batch of images is fed forward, the loss is calculated, and the weights are updated backward.

In one embodiment, training engine 201 evaluates the performance of the model (e.g., Naïve-CNN, VGG16) using metrics, such as accuracy, precision, recall, and F1-score (harmonic mean of a model's precision and recall).

In one embodiment, training engine 201 fine-tunes the deep convolutional neural network model for optimal performance. For example, training engine 201 adjusts the hyperparameters (e.g., learning rate, batch size, number of layers) or applies advanced techniques, such as transfer learning, to optimize the model. In one embodiment, training engine 201 retrains the model to improve emoticon performance. In one embodiment, the resulting trained deep convolutional neural network model is incorporated into an application, such as an iOS application.

In one embodiment, training engine 201 trains the deep convolutional neural network model to identify an emotion from images of facial expressions based on a sample data set, which may include labeled emotions for various images of facial expressions. In one embodiment, such labeled emotions (sample data set) are obtained from an expert or a pre-trained FER model. In one embodiment, the sample data set includes images of individuals expressing seven emotions photographed from five different angles.

Furthermore, in one embodiment, the sample data set discussed above is referred to herein as the “training data,” which is used by a machine learning algorithm to make predictions or decisions, such as identifying an emotion from images of facial expressions. The algorithm iteratively makes predictions on the training data until the predictions achieve the desired accuracy as determined by an expert. Examples of such learning algorithms include neural networks.

In step 302, capturing engine 202 captures an image of an individual from computing device 101, which, in one embodiment, corresponds to a mobile computing device.

As discussed above, in one embodiment, capturing engine 202 manages the camera feed of the computing device's (e.g., mobile computing device) camera hardware and saves the resulting image data.

In one embodiment, capturing engine 202 uses a framework (e.g., AVFoundation framework), which provides the necessary application programming interfaces (APIs) for interacting with the computing device's cameras.

In one embodiment, capturing engine 202 determines which camera on computer/computing device 101 (e.g., mobile computing device) to use. For facial emotion recognition (FER), in one embodiment, capturing engine 202 selects the front-facing camera to capture the individual using computing device 101.

In one embodiment, capturing engine 202 establishes a capture session using the AVFoundation framework, linking the selected camera input to a data output.

In one embodiment, capturing engine 202 displays a live camera feed on the computing device's screen (e.g., within the app interface). This allows the user to position their face correctly within the frame for optimal analysis.

In one embodiment, capturing engine 202 then captures a still image or extracts a video frame from the live stream.

In one embodiment, capturing engine 202 delivers the captured image as digital data (e.g., a UIImage object or raw pixel buffer) to analysis engine 203.

In one embodiment, capturing engine 202 uses a specific camera-related function (e.g., capturePhoto(with:delegate:) in iOS) to grab the image data. The resulting image is then cropped or processed to isolate the face before being passed to the trained Deep Convolutional Neural Network (DCNN) for analysis.

In step 303, analysis engine 203 analyzes the captured image of the individual, which, in one embodiment, corresponds to an individual on the autism spectrum or an individual that is neurotypical, using the trained deep convolutional neural network.

As stated above, in one embodiment, analysis engine 203 performs pre-processing and formatting. In one embodiment, the raw image captured by the camera is converted into a specific format and size required by the trained deep convolutional neural network model.

In one embodiment, analysis engine 203 receives the captured image data (e.g., a UIImage) from capturing engine 202.

In one embodiment, before emotion analysis, analysis engine 203 identifies the location of the face within the image. In one embodiment, such an individuation involves analysis engine 203 using a separate, fast face detection algorithm to output bounding box coordinates for the face.

In one embodiment, analysis engine 203 crops the image to focus only on the face. In one embodiment, analysis engine 203 resizes the cropped image to be the exact input dimensions that the trained deep convolutional neural network model expects (e.g., input dimension in pixels).

In one embodiment, analysis engine 203 normalizes (scales) the pixel values of the resized image to match the range of values used during the model's training phase (e.g., scaling pixel values from 0 to 255 to a floating-point range, such as 0 to 1 or −1 to 1).

In one embodiment, analysis engine 203 loads the pre-trained and optimized deep convolutional neural network into memory of computing device 101. On iOS devices, analysis engine 203 performs such a task using a machine learning framework, such as Core ML (for model integration and inference) or metal performance shaders (for hardware-accelerated computations).

In one embodiment, analysis engine 203 feeds the pre-processed, formatted image data into the deep convolutional neural network. In one embodiment, analysis engine 203 performs all the forward passes through the network's layers (convolutional, pooling, fully connected) as defined during the training process.

In one embodiment, the deep convolutional neural network produces an output vector from its final (softmax) layer. In one embodiment, such a vector contains a probability score for each of the seven universal emotion classes (e.g., happy, sad, angry, fearful, neutral, surprised, and disgust).

In one embodiment, analysis engine 203 interprets the model's output to prepare for classification. In one embodiment, analysis engine 203 reads the probability vector to determine the emotional state. For example, if the seven emotions are ordered as (anger, disgust, fear, happy, sad, surprise, neutral), and the vector is (0.02, 0.01, 0.03, 0.90, 0.01, 0.02, 0.01), then analysis engine 203 identifies happy as the most likely emotion with a 90% confidence.

In step 304, classification engine 204 classifies the emotional state of the individual based on the analysis of the captured image of the individual using the trained deep convolutional neural network.

As discussed above, in one embodiment, analysis engine 203 passes the prediction (both the chosen emotion and its confidence score) to classification engine 204.

In one embodiment, classification engine 204 converts the deep convolutional neural network model's output into a discrete, human-interpretable emotion label.

In one embodiment, classification engine 204 identifies the output vector. In one embodiment, the deep convolutional neural network's final layer (the softmax layer) produces an output vector (a list of numbers) where each value corresponds to the probability of the image belonging to one of the target emotion classes (e.g., happy, sad, angry, etc.). The sum of all probabilities in this vector equals 1.0 (or 100%).

For example, a seven-element output vector might look like this:
Output Vector=[0.05(angry), 0.92(happy), 0.01(sad), 0.02(neutral), . . . ]

In one embodiment, classification engine 204 uses the argmax (argument of the maximum) function for classification. In one embodiment, the argmax function finds the index of the highest probability value in the output vector. Classification engine 204 then iterates through the vector and identifies the class associated with the maximum probability.

In the example above, the highest probability is 0.92, which corresponds to the happy class. Therefore, the classified emotional state is happy.

In one embodiment, classification engine 204 implements a confidence threshold to handle ambiguous or uncertain classifications. For example, if the maximum probability (Pₘₐₓ) meets or exceeds a defined threshold (e.g., 75%), then the corresponding emotion is confidently classified. In another example, if the maximum probability is below the threshold, the classification is flagged as ambiguous or uncertain (e.g., “cannot determine emotion”), rather than making a low-confidence guess.

In one embodiment, classification engine 204 outputs the final output of this stage, which is a single, discrete, text-based or numerical label (e.g., “happy”) that represents the emotional state.

In step 305, classification engine 204 conveys the classified emotional state to a user, which, in one embodiment, corresponds to an individual on the autism spectrum or an individual that is neurotypical, of computing device 101 via an emoticon.

As stated above, in one embodiment, classification engine 204 conveys the classified emotional state to a user of computing device 101 via an emoticon by utilizing a mapping and display process, which may be handled by an application's user interface (UI) module.

In one embodiment, classification engine 204 maps the output (e.g., a discrete emotional state label, such as “happy”) to a corresponding visual element (the emoticon). In one embodiment, such a mapping is stored in a data structure, referred to herein as the “emoticon mapping table.” In one embodiment, such a data structure is stored in a storage device (e.g., storage device 113, 115) of computing device 101. In one embodiment, the data structure is populated by an expert.

In one embodiment, a dictionary or key-value array is used to pair the text-based classification with a specific emoticon or image asset. For example, the classified emotional state (input) is paired with an emoticon/asset (output). For instance, “happy” is paired with an emoticon of a happy graphic, “sad” is paired with an emoticon of a sad graphic, “angry” is paired with an emoticon of an angry graphic, “fearful” is paired with an emoticon of a fearful graphic, “neutral” is paired with an emoticon of a neutral graphic, “surprised” is paired with an emoticon of a surprised graphic, “disgust” is paired with an emoticon of a disgusted graphic, etc.

In one embodiment, the application's user interface component on computing device 101 is responsible for taking the selected emoticon and displaying it to the user of computing device 101. For example, the user interface component receives the classified state (e.g., happy) from classification engine 204. The user interface component then queries the emoticon mapping table to retrieve the corresponding visual asset (e.g., the code for the corresponding emoticon or the file path for the emoticon). In one embodiment, the application on computing device 101 then dynamically updates a designated area of the computing device's display (the user interface) by rendering the area with the retrieved emoticon.

In this manner, emotions of individuals, including individuals on the autism spectrum, are identified.

The benefits of utilizing such technology include being able to analyze complex data from facial expressions with high accuracy in real-time.

Furthermore, in one embodiment, the principles of the present disclosure operate via a mobile application, making it non-invasive and easily accessible to a wide user base. This ease of use extends to everyday environments, making continuous support in real-world settings practical.

Additionally, in one embodiment, the principles of the present disclosure address the challenges autistic individuals face by having the application aid in recognizing and understanding emotional cues, a critical area of need.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A computer-implemented method for emotion recognition, the method comprising:
training a deep convolutional neural network to identify an emotion from images of facial expressions;
capturing an image of an individual from a computing device;
analyzing said captured image of said individual using said trained deep convolutional neural network;
classifying an emotional state of said individual based on said analysis of said captured image of said individual using said trained deep convolutional neural network; and
conveying said classified emotional state to a user of said computing device via an emoticon.

2. The method as recited in claim 1, wherein said deep convolutional neural network is trained on a sample data set comprising images of individual expressing seven emotions photographed from five different angles.

3. The method as recited in claim 2, wherein said deep convolutional neural network comprises one of the following; Naïve-CNN, VGG16, EfficientNetV2, and MobileNetV2.

4. The method as recited in claim 1, wherein said computing device comprises a mobile computing device.

5. The method as recited in claim 1, wherein said individual corresponds to an individual on an autism spectrum.

6. The method as recited in claim 1, wherein said individual corresponds to an individual that is neurotypical.

7. The method as recited in claim 1, wherein said user corresponds to an individual on an autism spectrum.

8. The method as recited in claim 1, wherein said user corresponds to an individual that is neurotypical.

9. A computer program product for emotion recognition, the computer program product comprising one or more computer readable storage mediums having program code embodied therewith, the program code comprising programming instructions for:
training a deep convolutional neural network to identify an emotion from images of facial expressions;
capturing an image of an individual from a computing device;
analyzing said captured image of said individual using said trained deep convolutional neural network;
classifying an emotional state of said individual based on said analysis of said captured image of said individual using said trained deep convolutional neural network; and
conveying said classified emotional state to a user of said computing device via an emoticon.

10. The computer program product as recited in claim 9, wherein said deep convolutional neural network is trained on a sample data set comprising images of individual expressing seven emotions photographed from five different angles.

11. The computer program product as recited in claim 10, wherein said deep convolutional neural network comprises one of the following; Naïve-CNN, VGG16, EfficientNetV2, and MobileNetV2.

12. The computer program product as recited in claim 9, wherein said computing device comprises a mobile computing device.

13. The computer program product as recited in claim 9, wherein said individual corresponds to an individual on an autism spectrum.

14. The computer program product as recited in claim 9, wherein said individual corresponds to an individual that is neurotypical.

15. The computer program product as recited in claim 9, wherein said user corresponds to an individual on an autism spectrum.

16. The computer program product as recited in claim 9, wherein said user corresponds to an individual that is neurotypical.

17. A system, comprising:
a memory for storing a computer program for emotion recognition; and
a processor connected to the memory, wherein the processor is configured to execute program instructions of the computer program comprising:
training a deep convolutional neural network to identify an emotion from images of facial expressions;
capturing an image of an individual from a computing device;
analyzing said captured image of said individual using said trained deep convolutional neural network;
classifying an emotional state of said individual based on said analysis of said captured image of said individual using said trained deep convolutional neural network; and
conveying said classified emotional state to a user of said computing device via an emoticon.

18. The system as recited in claim 17, wherein said deep convolutional neural network is trained on a sample data set comprising images of individual expressing seven emotions photographed from five different angles.

19. The system as recited in claim 18, wherein said deep convolutional neural network comprises one of the following; Naïve-CNN, VGG16, EfficientNetV2, and MobileNetV2.

20. The system as recited in claim 17, wherein said computing device comprises a mobile computing device.

21. The system as recited in claim 17, wherein said individual corresponds to an individual on an autism spectrum.

22. The system as recited in claim 17, wherein said individual corresponds to an individual that is neurotypical.

23. The system as recited in claim 17, wherein said user corresponds to an individual on an autism spectrum.

24. The system as recited in claim 17, wherein said user corresponds to an individual that is neurotypical.
